(19) United States
(12) Patent Application Publication
(10) Pub. No.: US 2024/0366193 A1
(43) Pub. Date: Nov. 7, 2024

(54) SYSTEMS AND METHODS FOR STOOL CHARACTERIZATION AS NON-INVASIVE MEASURES OF DISEASE ACTIVITY

(71) Applicants: CEDARS-SINAI MEDICAL CENTER, Los Angeles, CA (US); DIETA INC., Oak Park, CA (US)

(72) Inventors: Gil Y. Melmed, Los Angeles, CA (US); Asaf Kraus, Oak Park, CA (US)

(21) Appl. No.: 18/656,278

(22) Filed: May 6, 2024

Related U.S. Application Data
(60) Provisional application No. 63/464,306, filed on May 5, 2023.

(51) Int. Cl. A61B 10/00; G16H 10/60

(52) U.S. Cl. CPC A61B 10/0038; G16H 10/60

(57) ABSTRACT

A method includes receiving one or more stool images from a client device; determining, from the one or more stool images, one or more stool characteristics; and providing disease activity information to the client device, based at least in part on analyzing the one or more stool characteristics.

### CROSS-REFERENCE TO RELATED APPLICATIONS

This application claims the benefit of and priority to U.S. Provisional Application No. 63/464,306, filed May 5, 2023, which is hereby incorporated by reference herein in its entirety.

### FIELD OF THE INVENTION

The present invention relates generally to assessing stool characteristics, and more specifically, to capturing images and/or videos of bowel movements to determine disease activity associated with inflammatory bowel disease.

### BACKGROUND

Over three million patients suffer from inflammatory bowel disease in the U.S. alone. Physicians, researchers, universities, hospitals, companies, etc., are involved in clinical trials to study efficacy of medical therapies associated with inflammatory bowel disease. Furthermore, hospitals and health systems provide inpatient services for at-risk patients whom they assume risk. Payers and insurers backstop costs for some patients and may want to know treatment efficacy for specific patients after these patients start a new drug. Easily monitoring patients, whether in-patient or out-patient, can provide caretakers and payers with information to adjust strategy if a specific medical therapy is not working. Early intervention can improve patient health and conserve social resources.

### SUMMARY OF THE INVENTION

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to certain aspects of the present disclosure, a method includes receiving one or more stool images from a client device. The method further includes determining, from the one or more stool images, one or more stool characteristics. And, based at least in part on analyzing the one or more stool characteristics, providing disease activity information to the client device.

According to certain aspects of the present disclosure, a system includes one or more data processors and a non-transitory computer-readable storage medium containing instructions which, when executed on the one or more data processors, cause the one or more data processors to perform operations including: (a) receiving one or more stool images, (b) determining, from the one or more stool images, one or more stool characteristics, and (c) providing disease activity information to the client device, based at least in part on analyzing the one or more stool characteristics.

According to certain aspects of the present disclosure, a system includes one or more data processors and a non-transitory computer-readable storage medium containing instructions which, when executed on the one or more data processors, cause the one or more data processors to perform operations including: (a) receiving a plurality of stool images from plurality of client devices, (b) determining, from the plurality of stool images, one or more stool characteristics associated with each of the plurality of client devices, (c) determining disease activity information, based at least in part on analyzing the one or more stool characteristics, (d) generating a corresponding report associated with a corresponding client device, wherein a corresponding file size associated with the corresponding report is proportional to a number of images associated with the corresponding client device, (e) based on the corresponding file size exceeding a threshold, generating an alert to a caregiver device.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments, and are therefore not to be considered as limitations on the scope of the various embodiments or claims.

FIG. 1 is a block diagram of a system for providing disease activity information, according to certain aspects of the present disclosure.

FIG. 2 is a process for providing disease activity information, according to certain aspects of the present disclosure.

FIG. 3 is a table of example results of stool characteristics, according to certain aspects of the present disclosure.

FIG. 4 is a graph comparing C-reactive protein values in stool samples that contain mucous and those that do not.

FIG. 5 is a graph comparing C-reactive protein values in stool samples that contain blood and those that do not.

FIG. 6A provides photographs of representative stool images for classifying mucus amount.

FIG. 6B provides photographs of representative stool images for classifying blood amount.

### DETAILED DESCRIPTION

Patients with ulcerative colitis (UC) are often asked to visually assess stool characteristics as a measure of disease activity. However, stool characteristics have not been validated against objective inflammation. A previous study found that trained artificial intelligence (AI) via smartphone application can measure certain stool characteristics in patients with irritable bowel syndrome. Current diagnosis of inflammatory bowel disease (IBD) requires a colonoscopy in patients for whom there is a high index of suspicion. Non-invasive tests to evaluate inflammation are inconvenient (e.g., blood and stool tests involve the need for obtaining blood draws or stool samples). There is a need to determine whether stool characteristics measured by AI and physicians correlate with inflammation in ulcerative colitis and other IBD. A photo would be a more convenient, efficient, and painless way to determine whether a colonoscopy is necessary for the diagnosis of IBD.

Some implementations of the present disclosure use a smartphone-based application (or app) to capture images, photos, or videos of stool samples. The captured images are analyzed using an artificial intelligence (AI) algorithm. The analysis includes diagnosing/prognosticating inflammatory bowel disease (IBD) activity (e.g., ulcerative colitis or Crohn's disease). Training process for the algorithm leverages information learned by physicians. For example, feedback from physicians that have reviewed and discussed various stool characteristics of stool samples as obtained by patients using the app can be used to train the algorithm. These stool characteristics include novel characteristics specific to IBD as well as previously identified characteristics. Previously identified characteristics include stool form (e.g., Bristol Stool scale), fuzziness, fragmentation, and volume. Novel IBD-specific characteristics include stool color, blood, mucous, and whether the photo was obtained with the feces in toilet water or not in water (i.e., a bedside commode), and whether that influences the AI interpretations.

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

Management of acute, severe ulcerative colitis (ASUC) in hospitals warrants daily assessment and evaluation of disease activity to determine the appropriateness of continuing corticosteroids, initiating rescue therapy, or surgery. Daily changes in patient-reported rectal bleeding, stool frequency and form assist with clinical decision-making in the context of objective changes in inflammatory markers but may be subject to interpretation and recall bias. Smart toilet technology has been used to mitigate recall bias, however smart toilets requires installation of special technology into existing plumbing. Some embodiments of the present disclosure can provide clinical utility for artificial intelligence applied to stool images acquired via smartphones for monitoring patients hospitalized with ASUC.

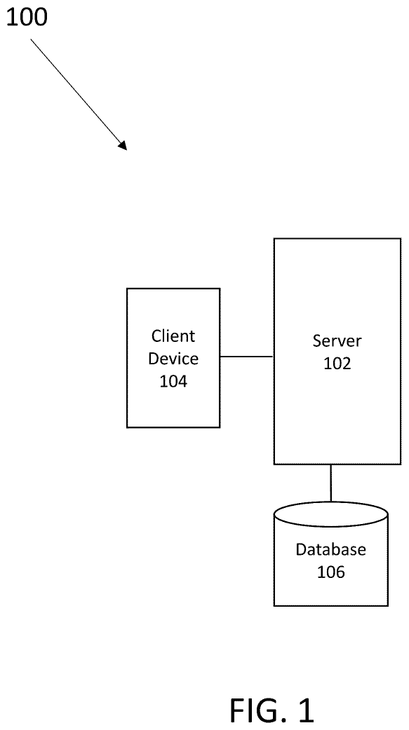

Referring to FIG. 1, a system 100 for determining disease activity information is provided, according to some implementations of the present disclosure. The system 100 includes a client device 104, a server 102, and a database 106. Each of these components can be realized by one or more computer devices and/or networked computer devices. The one or more computer devices include at least one processor with at least one non-transitory computer readable medium. In some implementations, the client device 104 is a smartphone with a camera for capturing images. In some implementations, the server 102 stores an AI algorithm for analyzing the captured images. In some implementations, the database 106 includes files, images, computational models, etc., used by the server 102 for analyzing the captured images. In some implementations, the client device 104 further includes a second smartphone used by a caregiver of the user of the smartphone.

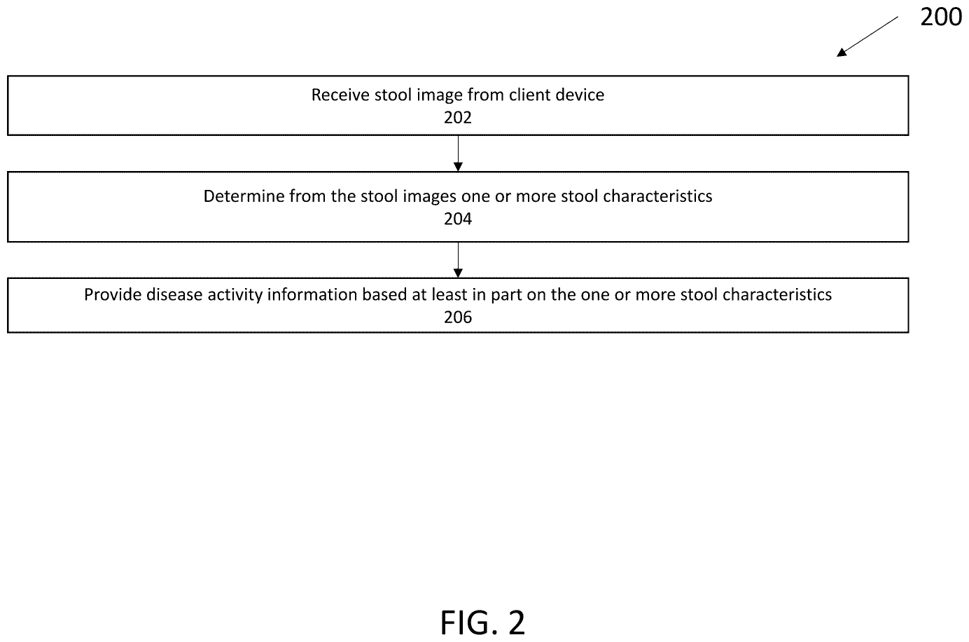

Referring to FIG. 2, a process 200 for providing disease activity information to a client device (e.g., the client device 104) is provided, according to some implementations of the present disclosure. At step 202, the server 102 receives one or more stool images from the client device 104. The client device 104 captures the one or more stool images using a camera associated with the client device 104. The one or more stool images can be captured in a wet environment (e.g., in a toilet) or in dry environment (e.g., in a commode).

In some implementations, the client device 104 captures the one or more stool images using one or more cameras integrated in the client device 104. In some implementations, the one or more stool images are captured by a separate camera and the stool images are stored a memory associated with the client device 104 (e.g., an internal memory of the client device 104, an external memory associated with the client device 104, the database 106, etc.). In some implementations, the client device 104 includes a first smartphone and a second smartphone. The first smartphone captures the stool images and shares the stool images with the second smartphone, and the second smartphone sends the stool images to the server 102.

In some implementations, the one or more stool images are received from multiple client devices. The server 102 can standardize each image received. For example, the server 102 can crop the image to a specific aspect ratio (e.g., a widescreen aspect ratio, a 1:1 aspect ratio, etc.), reduce a file size of the image to be under a file size limit (e.g., below a 3 MB file size, below 6 MB file size, etc.), convert a file type of the image from a received file type to a preferred file type (e.g., converting a PNG image file to a JPG image file), etc.

At step 204, the server 102 determines from the stool images one or more stool characteristics. In some implementations, the server 102 determines whether the stool provided in the one or more stool images is in a wet environment or a dry environment. The one or more stool characteristics determined include stool form (e.g., Bristol Stool scale), fuzziness, fragmentation, volume, stool color, blood, mucous, or any combination thereof.

In some implementations, the server 102 includes an AI model for determining the one or more stool characteristics. The AI model can provide one or more of the following stool characteristics: a Bristol stool scale associated with one or more of the stool images, a consistency associated with one or more of the stool images, edge fuzziness associated with one or more of the stool images, fragmentation associated with one or more of the stool images, volume associated with one or more of the stool images, blood amount associated with one or more of the stool images, mucus amount associated with one or more of the stool images, or any combination thereof. In some implementations, the AI model is a convolutional neural network, deep learning model, large language model, machine learning model, etc.

In some implementations, the different metrics provided by the AI model are provided on a scale. For example, Bristol stool scale can be provided as one of a Type 1 to a Type 7 on the scale. The Bristol stool scale can further be segmented where the different types including Type 1 to Type 7 are grouped such that Types 3 to 5 indicate ideal while the other types indicate an issue. In another example, consistency may be provided on a scale of 0 to 100, where 0 indicates a liquid consistency 100 indicates a solid consistency. In another example, edge fuzziness may be provided on a scale of 0 to 100, where 0 indicates very clear and 100 indicates very fuzzy. In another example, fragmentation may be provided on a scale of 0 to 100, where 0 indicates a single piece and 100 indicates many pieces.

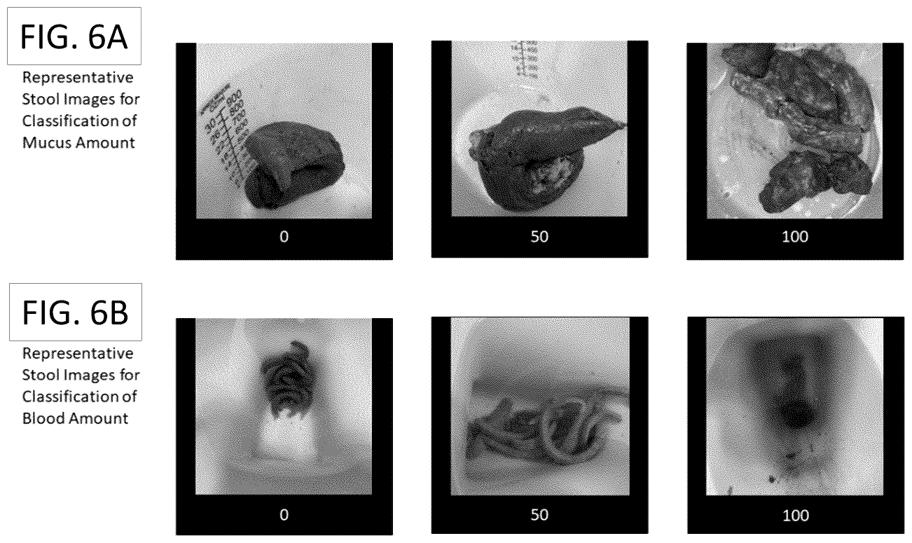

In another example, volume may be normalized to a standard volume (e.g., 0.5 pounds). In another example, volume may be provided on a scale of 0 to 100, where 0 indicates a very small volume and 100 indicates a very large volume. In another example, blood amount may be determined based on coloration and can be cast as a percentage of the determined volume of the stool. In some cases, the blood amount may be cast as a binary value, whether there is blood or not. In some cases, the blood amount may be cast on a scale from 0 to 100 as in FIG. 6B, where 0 indicates no blood and 100 indicates a large amount of blood. In another example, mucus amount may be cast as a percentage of the determined volume of the stool. In some cases, the mucus amount may be cast as a binary value, whether there is mucus or not. In some cases, the mucus amount may be cast on a scale from 0 to 100 as in FIG. 6A, where 0 indicates no mucus and 100 indicates large amount of mucus.

In some implementations, additional information is collected for the patient associated with the one or more stool images. Additional information can include patient demographics and relevant clinical information. Examples of demographics includes age, gender, race, and ethnicity. In some implementations, in a hospital setting, on admission, vital signs, stool frequency, quantity of blood in stool, hemoglobin and erythrocyte sedimentation rate (ESR) can be recorded to establish Truelove and Witts Severity Index for ulcerative colitis. Serum C-reactive protein (CRP) can be measured on admission and/or daily while hospitalized. Fecal calprotectin, presence of enteric infections, treatment received, and length of hospitalization can be further recorded. In some implementations, colonoscopy or sigmoidoscopy with or without biopsies, or intestinal ultrasound, or computerized tomography, or magnetic resonance imaging can be further recorded.

At step 206, the server 102 provides disease activity information based at least in part on the one or more stool characteristics. The disease activity information can include disease activity information for all forms of IBD including ulcerative colitis (not just acute severe ulcerative colitis), Crohn's disease, pouchitis, microscopic colitis, etc.

In some implementations, at least one of the one or more stool characteristics is modified based on whether the stool image was captured in a wet environment or in a dry environment. For example, pictures might be obtained of stool submersed in water in a toilet, or of stool deposited in a commode without water.

In some implementations, the one or more stool characteristics can be used to determine whether a patient is suffering from IBD and whether a colonoscopy is necessary to clarify. One or more of the one or more stool characteristics can be positively correlated with IBD such that a presence of a combination of the one or more stool characteristics is indicative of the patient suffering from IBD. In some implementations, the server 102 has access to specific statistical correlations between a stool characteristic and contribution to determining whether the patient is suffering from IBD. In some implementations, if mucus is present (i.e., if a binary value indicates the presence of mucus), then the server 102 can determine that a colonoscopy is necessary to clarify an IBD status of the patient.

In some implementations, if a patient is known to suffer from IBD, the one or more stool characteristics can be used to determine whether there is a flare-up. Among patients with IBD, their disease waxes/wanes, and it is not always clear that symptoms (i.e., diarrhea, change is stool habits) represent increased disease activity (which may then require changes to medical therapy) or non-IBD causes.

In some implementations, if a patient is known to suffer from ulcerative colitis which is flaring and the patient is in the hospital, the one or more stool characteristics can be used to determine whether medical therapy is improving, deteriorating, or having no effect on the patient's condition. In some implementations, the one or more stool characteristics can be used to inform on a likelihood the patient may require surgery. Among patients with IBD hospitalized with ulcerative colitis (or certain forms of Crohn's disease), the risk of colectomy is very high (over 30% during that admission). Patients are evaluated day-to-day in the hospital by medical and surgical teams, and the decisions to go to surgery are often prolonged due to lack of clarity as to whether medical therapy is working or not. Thus, the one or more stool characteristics can provide information associated with disease activity to help discriminate those patients who are improving from those who are not and that should anticipate surgery.

In some implementations, a physician or researcher conducting a clinical trial to determine efficacy and/or response to treatment or medical therapy can use provided disease activity information to assess therapeutic efficacy. Typically, multiple colonoscopies are required in order to determine the efficacy/response to treatment. In a clinical trial setting, colonoscopies for the sake of assessments are cumbersome. Thus, obtaining accurate disease activity information in a non-invasive manner may help provide easier ways to assess therapeutic efficacy.

In some implementations, the server 102 maintains a history of determinations of disease activity information for the patient. As such, over time, the patient can be monitored to develop a trend of whether particular treatments are effective or whether the particular treatments need to be tweaked. In some implementations, the server 102 can provide a change in diet recommendation to the client device 104. In some implementations, the server 102 can alert a caregiver of the patient to provide a change in therapy or treatment to the patient.

Furthermore, the server 102 can store stool images associated with the patient in the database 106 for further analysis. Disease activity information is an ongoing monitoring of how the disease is being managed over time, for example, whether the diseased state of the patient is improving, staying the same, or deteriorating. That is, the disease activity information is referenced to a specific time period.

In some implementations, the disease activity information is captured in a report. Each instance in the report can be associated with a specific patient where stool images in the report are limited to a specific file size and/or a specific aspect ratio along with an indication of the determined disease activity information. With such limitations on instances in the report, the size of the report is predictable and standardized such that as more stool instances are added to the report. The file size associated with each report along with a period of time that a patient has been monitored can be used as a gauge to whether the patient is having too frequent bowel movements or not enough bowel movements. In some implementations, the file size associated with the report is used to generate alerts for a caregiver to check the progression of disease activity information for the patient. For example, disease activity information may provide a trend after two, three, four, five, or six bowel movements, and the file size of the report can be used to check whether the server 102 has generated enough information for a threshold amount of bowel movements for the caregiver to check progression of disease activity information.

Examples are provided below for characterizing stool samples according to some implementations of the present disclosure.

Patients hospitalized with acute severe ulcerative colitis (ASUC) were asked to capture images of all bowel movements using a smartphone application. Validated AI was used to measure stool characteristics, including Bristol stool scale, consistency, edge fuzziness, fragmentation, and volume. Additionally, four physicians, including an inflammatory bowel disease specialist, scored each image for blood amount, mucus amount and whether stool was in a toilet or commode. Serum C-reactive protein (CRP) was measured daily, and each bowel movement was associated with a CRP value obtained within 12 hours of the bowel movement. AI measurements and mean physician scores were rank normalized and correlated with rank normalized CRP values using mixed linear regression models. Mann-Whitney tests were used to compare median CRP values of images with and without mucus.

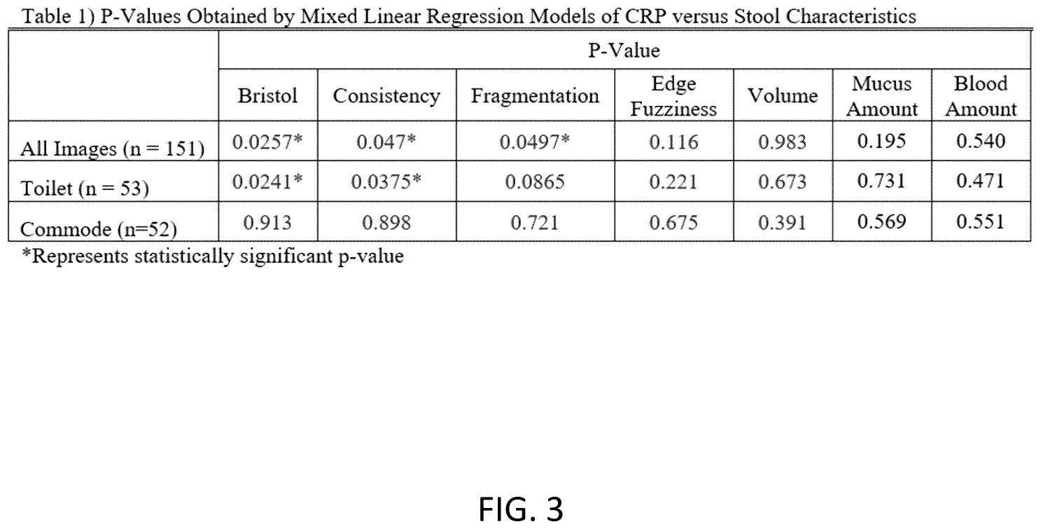

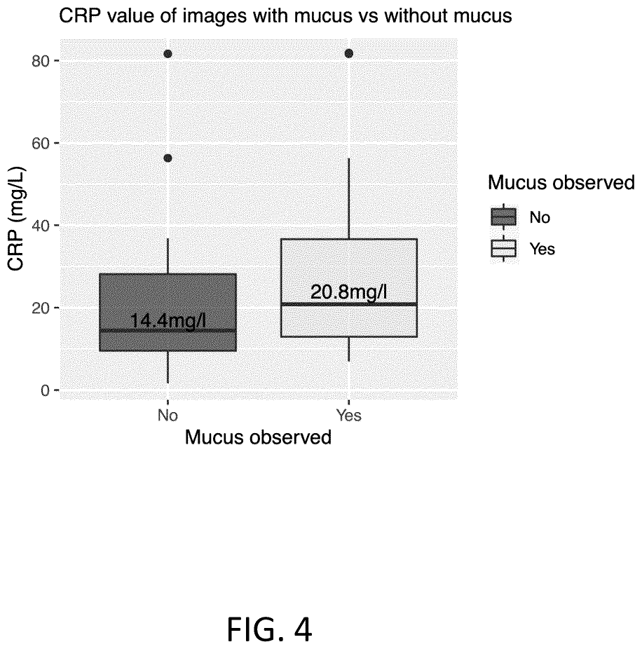

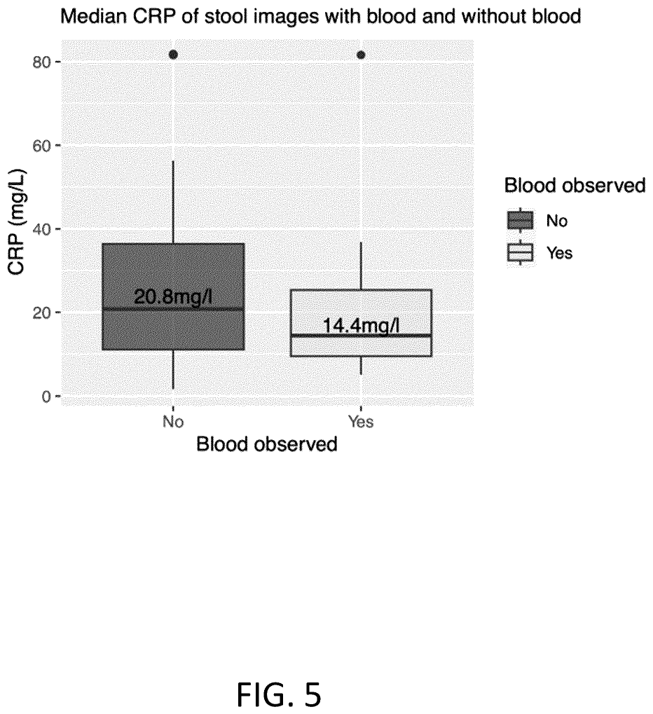

151 stool images were analyzed and collected from five patients admitted with ASUC (mean age 42 years, 40% male). 53 images were capture in a toilet, 52 images were in a bedside commode, and 46 were in a commode but were somewhat obscured by urine. Overall, Bristol stool scale and fragmentation positively correlated with CRP (p=0.026 and 0.049, respectively), while consistency negatively correlated with CRP (p=0.047). Volume, edge fuzziness, mucus amount and blood amount did not correlate with CRP. When analyzing toilet images alone, Bristol and consistency correlations remained significant (p=0.024 and 0.038), but these correlations were not seen when analyzing unobscured commode images alone. The median CRP of images with mucus was higher than that of images without mucus (p=0.011). These results are summarized provided in FIG. 3. Additionally, the median CRP corresponding to images without blood was marginally higher than that of images with blood (p=0.07502). The significant correlations indicate that stool images analyzed for the listed characteristics can predict inflammatory activity and thus serve as a noninvasive marker of disease activity. FIG. 4 provides a comparison of CRP values of images with mucous and those without. FIG. 5 provides a comparison of CRP values of images with blood and those without.

Demographics and Clinical Information that was obtained is summarized in Table 2.

TABLE 2
Demographics and Clinical Information

| Sex | | |
|---|---|---|
| Male | 40% | |
| Female | 60% | |
| Race | | |
| White | 80% | |
| Asian | 20% | |
| Ethnicity | | |
| Hispanic | 40% | |
| non-Hispanic | 60% | |
| Truelove-Witts Severity | 40% moderate, 60% severe | |
| Age (Mean ± SD) | 41.8 ± 20.66 | years |
| Number of daily bowel movements on | 16.4 ± 6.11 | |
| admission (Mean ± SD) | bowel movements | |
| Temperature on admission (Mean ± SD) | 98 ± 0.61° | F. |
| Heart rate on admission (Mean ± SD) | 89.8 ± 19.98 | bpm |
| Hemoglobin on admission (Mean ± SD) | 13.68 ± 1.79 | g/dL |
| Erythrocyte sedimentation rate (ESR) on | 17.75 ± 5.05 | mm/hr |
| admission (Mean ± SD) | | |
| C-reactive protein (CRP) on admission | 51.46 ± 31.33 | mg/L |
| (Mean ± SD) | | |
| Fecal calprotectin on admission | 1684.8 ± 1741.12 | μg/g |
| (Mean ± SD) | | |
| Length of hospitalization (Mean ± SD) | 12.4 ± 6.66 | days |
| Number of stool photos obtained | 30.2 ± 38.92 | photos |

| | |
|---|---|
| (Mean ± SD) | |
| Patients with enteric infections | 1 campylobacter, 2 cytomegalovirus |
| Patients who received intravenous | 4 patients |
| corticosteroids and infliximab | |
| Patients who received other treatments | 1 azithromycin, 1 oral corticosteroids, 1 |
| | azathioprine and ganciclovir |

Smartphone application AI measurements of Bristol stool scale, stool consistency, and stool fragmentation significantly correlate with CRP values in hospitalized patients with ASUC. Additionally, median CRPs are higher when mucus is seen. Further training of smartphone-based AI algorithms to validate the association of stool characteristics with objective inflammation can yield a novel, non-invasive tool for UC disease monitoring.

Embodiments of the present disclosure provide a system that can be used for IBD disease monitoring situations including acute severe UC or Crohn's disease in the hospital; routine monitoring in the ambulatory setting in times of symptoms or even without symptoms, after a change in therapy (or dose) to determine treatment response, at a time of diagnostic testing (i.e. blood tests or stool tests for inflammation, colonoscopy, or imaging (ultrasound, CT or MRI)) to correlate with noninvasive stool image capture.

Embodiments of the present disclosure provide a system that can be used clinically in several potential scenarios: (a) to facilitate diagnosis of ulcerative colitis or Crohn's disease, (b) to diagnose a ‘flare’ of ulcerative colitis or Crohn's disease, (c) to determine prognosis of hospitalized patients with ulcerative colitis or Crohn's disease, and (d) for use in clinical trials as a measure of disease activity (i.e., drug or device trials seeking to treat either Crohn's disease or ulcerative colitis).

Embodiments of the present disclosure provide a non-invasive approach with minimal inconvenience. That is, no need for bowel preparation, no need for blood draw, no need collect physical stool samples, etc. An image is enough for analysis.

Embodiments of the present disclosure provide a hospital a scalable solution for managing care and monitoring disease activity associated with patients afflicted with ASUC. An AI system can monitor specific bowel movements of patients for both in-patient and out-patient care. For example, the system 100 can maintain a central database for generating reports on various patients. Disease activity information from the various patients is generated at different time intervals, and the system 100 can determine, maintain and format the disease activity information as images are received from client devices. In some implementations, a doctor or caregiver at a hospital can be alerted to a change in disease activity information for a subset of patients associated with the doctor or caregiver. In some implementations, timing and frequency of when stool images are received and when the system 100 determines disease activity information for patients can differ. As the subset of patients associated with the doctor or caregiver provide enough stool images to reach a threshold for checking the report, an alert can be provided to the doctor or caregiver to check reports associated with the subset of patients.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A method comprising:
receiving one or more stool images from a client device;
determining, from the one or more stool images, one or more stool characteristics; and
providing disease activity information to the client device, based at least in part on analyzing the one or more stool characteristics.

2. The method of claim 1, wherein the one or more stool characteristics include stool form, fuzziness, fragmentation, volume, stool color, blood, mucous, or any combination thereof.

3. The method of claim 2, wherein the stool form is determined as the Bristol stool scale.

4. The method of claim 1, wherein the client device includes a first device and a second device, the first device associated with a patient and configured to send the one or more stool images to the second device associated with a caregiver, and wherein the one or more stool images is received from the second device.

5. The method of claim 1, wherein the disease activity information is referenced to a specific time period.

6. The method of claim 5, wherein the disease activity information indicates a flare up over the time period.

7. The method of claim 6, further comprising:
providing to the client device an adjustment to a therapy associated with managing the flare up.

8. The method of claim 1, further comprising:
providing to a caregiver an alert associated with the disease activity information.

9. The method of claim 1, wherein the disease activity information indicates ulcerative colitis or Crohn's disease activity information.

10. A system, comprising:
one or more data processors; and
a non-transitory computer-readable storage medium containing instructions which, when executed on the one or more data processors, cause the one or more data processors to perform receiving one or more stool images,
determining, from the one or more stool images, one or more stool characteristics, and
providing disease activity information to a client device, based at least in part on analyzing the one or more stool characteristics.

11. The system of claim 10, wherein the one or more stool characteristics include stool form, fuzziness, fragmentation, volume, stool color, blood, mucous, or any combination thereof.

12. The system of claim 11, wherein the stool form is determined as the Bristol stool scale.

13. The system of claim 10, wherein the client device includes a first device and a second device, the first device associated with a patient and configured to send the one or more stool images to the second device associated with a caregiver, and wherein the one or more stool images is received from the second device.

14. The system of claim 10, wherein the disease activity information is referenced to a specific time period.

15. The system of claim 14, wherein the disease activity information indicates a flare up over the time period.

16. The system of claim 15, wherein the one or more data processors are further caused to perform operations including:
providing to the client device an adjustment to a therapy associated with managing the flare up.

17. The system of claim 10, wherein the one or more data processors are further caused to perform operations including:
providing to a caregiver an alert associated with the disease activity information.

18. The system of claim 10, wherein the disease activity information indicates ulcerative colitis or Crohn's disease activity information.

19. A system, comprising:
one or more data processors; and
a non-transitory computer-readable storage medium containing instructions which, when executed on the one or more data processors, cause the one or more data processors to perform receiving a plurality of stool images from plurality of client devices,
determining, from the plurality of stool images, one or more stool characteristics associated with each of the plurality of client devices,
determining disease activity information, based at least in part on analyzing the one or more stool characteristics,
generating a corresponding report associated with a corresponding client device, wherein a corresponding file size associated with the corresponding report is proportional to a number of images associated with the corresponding client device, and
based on the corresponding file size exceeding a threshold, generating an alert to a caregiver device.

20. The system of claim 19, wherein the caregiver device is included in the plurality of client devices.
